(19) United States
(12) Patent Application Publication
(10) Pub. No.: US 2006/0069433 A1
(43) Pub. Date: Mar. 30, 2006

(54) Intraocular lens

(75) Inventor: Yehoshua Ben Nun, Herzeliya (IL)

Correspondence Address: Harold L. Novick;NATH & ASSOCIATES PLLC, 6th Floor, 1030 15th Street, N.W., Washington, DC, 20005, US

(73) Assignee: NULENS, LTD.,

(21) Appl. No.: 11/281,353

(22) Filed: Nov. 18, 2005

Related U.S. Application Data
(63) Continuation of application No. 10/468,465, filed on Mar. 19, 2004.

(30) Foreign Application Priority Data
Feb. 20, 2001 (IL) 141529

(51) Int. Cl. A61F 2/16; A61F 9/013

(52) U.S. Cl. 623/6.45; 623/6.43; 606/107

(57) ABSTRACT

An intraocular lens system to be implanted in the posterior chamber of an eye, the system comprises a lens having an optical axis and at least two haptics extending from the circumference of the lens. The two haptics each includes one or more teeth located on their periphery.

### FIELD OF THE INVENTION

This invention relates to intraocular lens implantation and particularly to implantation in the posterior chamber of an eye.

### BACKGROUND OF THE INVENTION

Cataract is clouding of the natural lens of the eye or of its surrounding transparent membrane, which obstructs the passage of light causing various degrees of blindness. To correct this condition, a surgical procedure is known to be performed in which the opaque natural lens, or cataract, is extracted and replaced by an artificial intraocular lens.

The natural lens, located behind the iris in the posterior chamber in front of the vitreous cavity of the eye, is composed of a capsular bag containing gelatinous material. If this bag, called the posterior capsule, is left intact during a cataract extraction procedure, it may serve as a stable support site for implanting an intraocular lens. However, in the course of surgery, the posterior capsule may be inadvertently damaged or removed along with the cataract, in which case it would no longer be able to provide a support base to keep the intraocular lens from floating back into the vitreous cavity. In this case, it is known to implant the lens in the anterior chamber in front of the iris, or in the posterior chamber behind the iris, wherein the iris serves as a carrier for the lens in both instances. In the latter case, it has also been known to fix the intraocular lens in place behind the iris by suturing it to the ciliary sulcus. In both of the above cases, to maintain the intraocular lens properly centered, it is normally equipped with extensions, called haptics, which may have positioning holes to facilitate the centering of the lens.

U.S. Pat. No. 4,750,904 discloses a method of implanting an intraocular lens in the posterior chamber by tying the haptics to the iris and using small, radially disposed loops formed on the lens to serve as suture sites for securing the implanted lens directly to the iris.

### SUMMARY OF THE INVENTION

In accordance with the present invention, there is provided a novel solution for the self-fixation of an intraocular lens system in the posterior chamber of an eye. The lens system comprises a lens having an optical axis and at least two extending haptics attached to the circumference of the lens. These two haptics each have one or more teeth located on their periphery, which are particularly capable of penetrating the ciliary sulcus of the scleral wall of the eye, thereby anchoring the lens in place.

The teeth may be oriented to form an acute angle with the circumference of the haptic, thereby allowing free rotation of the haptic in one direction relative to the optical axis and allowing penetration of the teeth into the ciliary sulcus, when rotated in the other direction. The teeth may be harpoon-shaped, smooth or jagged in order to further facilitate their penetration or grasping of the ciliary sulcus.

The present invention provides for a secure self-attachment of the intraocular lens in the posterior chamber independently of the posterior capsule and without involving the iris.

### BRIEF DESCRIPTION OF THE DRAWINGS

In order to understand the invention and to see how it may be carried out in practice, different embodiments will now be described, by way of non-limiting examples only, with reference to the accompanying drawings, in which:

FIG. 1 is a schematic perspective view of an intraocular lens system according to the present invention;

FIG. 2 is a schematic view of the intraocular system shown in FIG. 1, when implanted in the posterior chamber of an eye;

FIGS. 3A, 3B, and 3C illustrate haptics of the lens system of FIG. 1, in accordance with three alternative embodiments of the present invention,

FIG. 4 is a schematic perspective view of an intraocular lens system according to another embodiment of the present invention;

FIG. 5 shows an optional supporting tool and a lens system according to the present invention, when supported by this tool for implantation;

FIG. 6 shows an optional protective sleeve for use with a lens system according to the present invention.

### DETAILED DESCRIPTION OF THE INVENTION

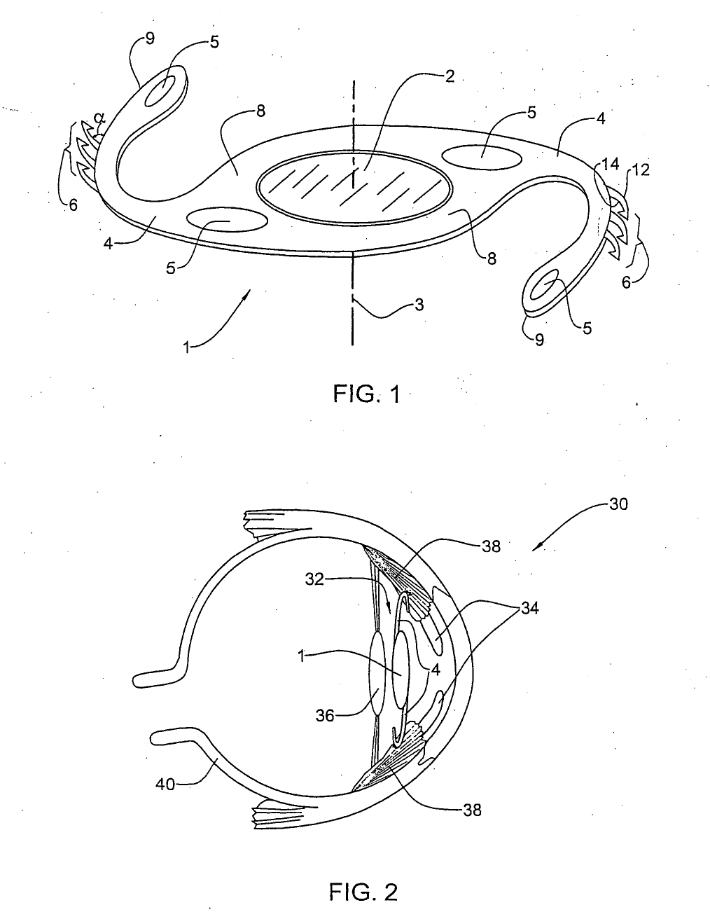

An intraocular lens system 1 in accordance with one embodiment of the present invention is shown in FIG. 1. The lens system 1 consists of a lens 2 of any known type having an optical axis 3 and two flexible haptics 4 extending away from the circumference of the lens 2. The haptics 4 include longitudinal positioning holes 5 near their base 8 and near their tip 9. These holes 5 aid in the positioning of the lens system 1 and facilitate manipulations thereof. The holes 5 may have any appropriate shape and each haptic 4 may have any number of them at any appropriate location.

With reference to FIG. 2, the lens system 1 is adapted to be implanted in an eye 30 to replace a cataract. FIG. 2 shows that, when implanted, the lens system 1 is located in the posterior chamber 32 between the iris 34 and the posterior capsule 36, with its haptics 4 bearing against the ciliary sulcus 38 of the scleral wall 40 of the eye 30.

In accordance with the present invention, and as seen in FIG. 1, each haptic 4 is provided with teeth 6 located at its periphery in the region designed to contact with the ciliary sulcus 38, when the lens system 1 is implanted in an eye. Thus, upon insertion of the lens system 1 into the eye 30 and manipulation of the haptics 4, the teeth 6 are made to penetrate and embed themselves in the ciliary sulcus 38 of the scleral wall 40, thereby securely anchoring the intraocular lens system 1 in the posterior chamber 32.

Reverting to FIG. 1, the teeth 6 may be harpoon-shaped with one smooth side 12 and one indented side 14, which is oriented to form an acute angle α with the circumference of the haptic 4. Such shape and orientation of the teeth 6 enable free rotation of the haptic 4 and the system 1 in one direction relative to the optical axis 3 and penetration and embedding of the teeth 6 into the ciliary sulcus 38, when rotation is attempted in the other direction.

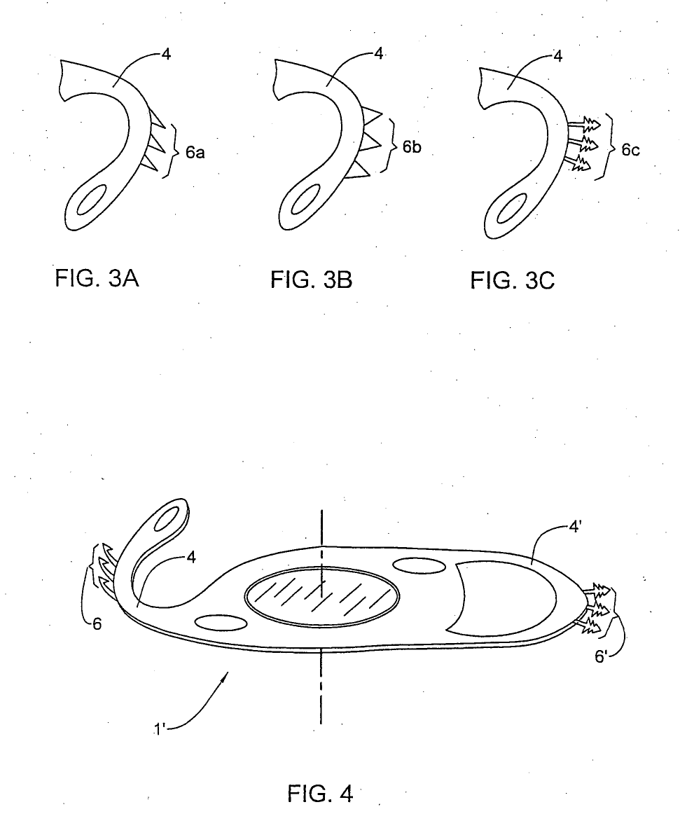

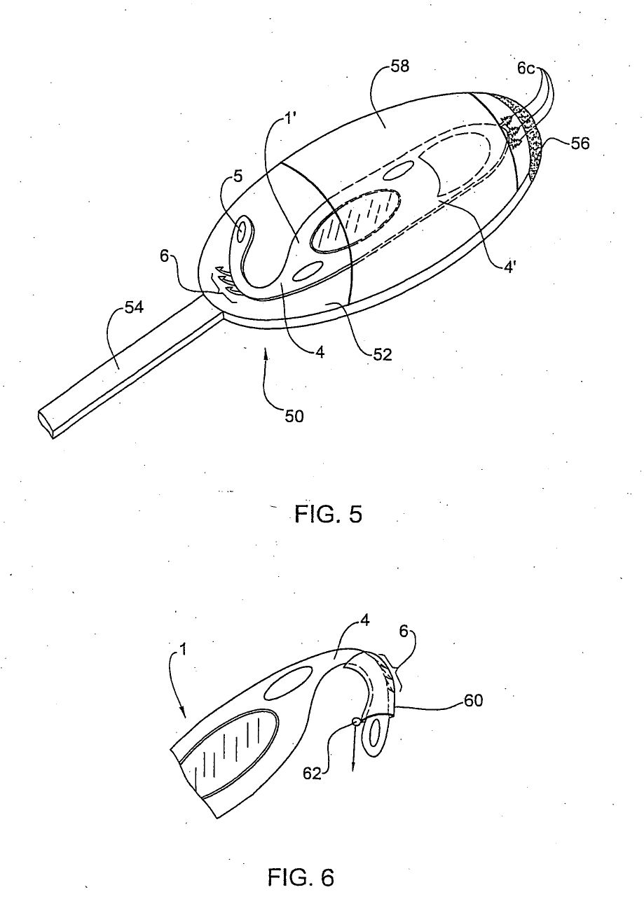

FIGS. 3A, 3B, and 3C show alternative designs for the teeth 6. In FIG. 3A, the teeth 6a are smooth on both sides and are acutely angled to allow rotation of the haptic 4 in only one direction. In FIG. 3B, the teeth 6b are similar to the teeth 6a but are oriented perpendicular to the circumference of the haptic 4 so as to prevent rotation in either direction. As shown in FIG. 3C, the teeth 6c are jagged to ensure an extremely firm anchoring of the haptics 4 in the eye 30.

To better secure the attachment of the lens system 1 and to center the lens 2, manipulation of the haptics 4 and the lens 2 can be performed by using the longitudinal positioning holes 5. It has been found that the longitudinal design of the positioning holes 5 is particularly useful as it allows for greater facility in the manipulation of the intraocular lens system 1 than conventional circular positioning holes. While oval positioning holes 5 are shown here, the longitudinal positioning holes 5 may be of various oblong shapes and sizes, with their length extending in the longitudinal direction of the haptic 4. Thus, rectangular or slit-like positioning holes may also be used. The oblong designs allow a surgeon to know when the intraocular lens system 1 has abutted the ciliary sulcus 38 during implantation.

In each haptic 4, 4′, any number of teeth 6 may be used and they may be placed anywhere on the far outer periphery of the haptics and at various distances from each other. The arrangement of the teeth on the two haptics of the lens system may be similar or completely different.

The lens 2 and haptics 4, 4′ may be produced as one body or alternatively, they may be produced as separate bodies, attachable to each other.

The teeth 6 may also be produced as one body along with the haptics 4, 4′ or rather they may be produced separately from the haptics, for example, to be attached thereto prior to implantation. In the latter case, it may be especially advantageous to produce a lens system to which teeth can be attached, when needed, and which can also be used without such teeth. For this purpose, the haptics should be formed with suitable teeth engagement means and/or the teeth may be formed with corresponding haptic engagement means.

The lens system of the present invention may be composed of various different substances. One example is making the teeth 6 of a biodegradable material, because it is beneficial to have the teeth 6 that penetrate the ciliary sulcus 38 completely dissolve over time. Another example is making the teeth 6 of a magnetic material so that they can be made to penetrate the sulcus 38 of the eye 30 by the use of an external magnet, after the haptic 4, 4′ has been positioned at the penetration site.

FIG. 5 illustrates an auxiliary tool 50 that may be used with an intraocular lens system of the present invention, for example, to facilitate its introduction into the eye 30. The auxiliary tool 50 may consist of a base 52 having a shape and dimensions to fully accommodate the lens system and a handle 54 attached to the base 52 at its end opposite its operative end 56. The tool 50 also has a covering member 58 to keep the lens system 1′ securely on the base 52 thereby restricting the motion of the lens system 1′ and reducing the likelihood of damaging the eye during implantation. While the auxiliary tool 50 should be rigid in order to direct the intraocular lens system 1′ into its operative position, its operative end 56 should preferably be soft and flexible to prevent damaging the eye. As an example of how this tool 50 can be used, an asymmetric intraocular lens system 1′ is shown thereon, having jagged teeth 6c on one haptic 4′ and harpoon-shaped teeth 6 on the other haptic 4. During its implantation, the lens system 1′ is brought to the ciliary sulcus 38 using the tool 50, and is pushed towards it, where the jagged teeth 6c are made to embed themselves firmly. The positioning holes 5 on the other haptic 4 are then used to embed the harpoon shaped teeth 6 at another desired site in the sulcus 38.

FIG. 6 shows an additional embodiment of the present invention, in which a thin protective sleeve 60 is placed over the teeth 6 so as to press them down to the haptic 4, thereby ensuring that they do not contact and damage the eye. The sleeve 60 should have a means for its removal from the haptic 4, such as an engaging loop 62, which can be pulled in the direction of the arrow to withdraw the sleeve 60 when the intraocular lens system 1 reaches the appropriate site of the ciliary sulcus 38, thereby exposing the teeth 6 and allowing them to penetrate the sulcus 38.

It should be understood that any permutation and/or combination of different features of the above-disclosed embodiments is also possible. It should further be understood that the above described embodiments constitute only examples of an intraocular lens system and a manner of its implantation according to the present invention, and that the scope of the present invention fully encompasses other embodiments which may become obvious to those skilled in the art.

1. An intraocular lens system to be implanted and securely anchored in a posterior chamber of an eye, the system comprising
a lens having an optical axis; and
a haptic system associated with said lens such that said lens can be retained in the eye said haptic system being securely implantable in an interior surface of that portion of a scleral wall that forms the outermost border of a ciliary sulcus and which scleral wall is comprised of tough connective tissue of the eye, said haptic system when so implanted in the eye firmly attaches said lens in an operative position
said haptic system comprising
at least two haptics, each haptic having an outer periphery and extending outwardly from a circumference of said lens, and at least three, spaced apart, penetrating members, each penetrating member having a tip portion and at least one penetrating member located on each of said haptic outer periphery, each penetrating member extending in a generally radially outward direction from said optical axis, said haptic and penetrating member having a shape and strength such that said penetrating member tip portion can penetrate the ciliary sulcus scleral wall so that said lens system is anchored in the posterior chamber of the eye.

2. An intraocular lens system according to claim 1, wherein at least one of said penetrating member tip portion is the most radially extreme point with respect to said optical axis.

3. An intraocular lens system according to claim 2, wherein at least one of said penetrating members is oriented to form an acute angle with a circumference of the haptic, so as to allow smooth rotation of the intraocular lens system in one direction relative to the optical axis and to enable penetration of the teeth into the ciliary sulcus scleral wall when rotated in the other direction.

4. An intraocular lens system according to claim 1, wherein at least one of said penetrating members has a harpoon-shaped outer surface.

5-6. (canceled)

7. An intraocular lens system according to claim 1, wherein said lens and said haptics are an integral, one piece body.

8-12. (canceled)

13. An intraocular lens system according to claim 10, wherein at least one of said penetrating members is made of a magnetic material.

14. An intraocular lens system according to claim 1, further including longitudinal positioning holes to facilitate the manipulation of the haptics and the lens.

15. (canceled)

16. An intraocular lens system according to claim 1, further including a rigid tool to support the lens system for directing it into its operative position.

17-18. (canceled)

19. An intraocular lens system according to claim 1, wherein said haptic system further includes a protective, removable sleeve located on at least one of said haptics for depressing said penetrating members thereon and removable upon implantation of said haptic system in an eye.

20-22. (canceled)

23. An intraocular lens system as claimed in claim 1 and further including a base that has a generally oblong shape in plan view and a circumference and to which said lens is securely attached; and
wherein said haptics are each flexible and elongate and are mounted at one end thereof to said base and together said haptics curve away from the base circumference in a spiral direction, each said haptic comprising an outer edge with respect to said base; and wherein at least one radially extending penetrating member is mounted on said outer edge of one haptic and is capable of curvilinear penetration upon rotation of said lens system about the visual axis of the eye in a first direction and is free to rotate about the eye visual axis in the other direction, and at least two spaced apart penetrating members are mounted on said outer edge of said other haptic and are capable of curvilinear penetration upon rotation of said lens system about the visual axis of the eye in said first direction and is free to rotate about the eye visual axis in said other direction.

24. A haptic system for an intraocular lens system which is to be implanted and securely anchored in a ciliary sulcus scleral wall in posterior chamber of an eye having an optical axis, said haptic system comprising
a base which can retain a lens that has an optical axis; and
at least two haptics extending radially outwardly from a circumference of said base, said two haptics together including at least three radially outwardly extending penetrating members, each haptic and penetrating member having a shape and strength so said penetrating member can penetrate the ciliary sulcus scleral wall and remain embedded therein so that said lens system is securely anchored in the eye independent of support from an eye posterior capsule and from an eye iris, whereby said haptic system when so implanted in the eye said lens is rigidly fixed in an operative position.

25. An intraocular lens system as claimed in claim 24 wherein at least one of said penetrating members is oriented to form an acute angle with a circumference of said haptic system base so as to allow smooth rotation of the intraocular lens system in one direction relative to the optical axis and to enable penetration of said penetrating members into the ciliary sulcus scleral wall when rotated in the other direction.

26. An intraocular lens system as claimed in claim 24 wherein at least one of said haptics has a leading end on which is mounted at least two penetrating members that are spaced apart so that said penetrating members can outwardly, radially with respect to the optical axis, penetrate into the tough connective tissue that form a part of the wall defining the ciliary sulcus for secure embedment therein for anchoring said haptic system in the ciliary sulcus of the eye.

27. An intraocular lens system as claimed in claim 24 wherein said haptic system includes a pair of oppositely directed haptics each having at least three penetrating members for outward radial penetration with respect to the eye's optical axis into the tough connective tissue of the scleral wall of the eye.

28. A method of implanting an intraocular lens in an eye, the method comprising,
inserting a haptic system having a first side and a second side into a posterior chamber of an eye having an optical axis, said haptic system comprised of at least a first and a second haptic on respective haptic system sides, each haptic having an outer periphery and at least one penetrating member extending outwardly from said outer periphery, the penetrating member having a shape and strength so that said penetrating member can penetrate a ciliary sulcus scleral wall of the eye and remain embedded therein;
positioning the haptic system such that at least said first haptic contacts the ciliary sulcus scleral wall of the eye;
manipulating the haptic system such that at least a penetrating member on said first haptic penetrates the ciliary sulcus scleral wall and is securely embedded therein so as to securely anchor said haptic in the ciliary sulcus of the eye;
positioning the haptic system further such that said other haptic if not already in contact with the ciliary sulcus scleral wall, contacts the ciliary sulcus scleral wall; and
manipulating the haptic system such that a penetrating member on said second haptic if not already penetrating the ciliary sulcus scleral wall penetrates the ciliary sulcus scleral wall and is securely embedded therein so as to securely anchor said haptic system in the posterior chamber of the eye.

29. The method as claimed in claim 28 wherein said haptic system further comprises a base having a circumference; and wherein each said haptic is flexible and elongate and is mounted at one end thereof to said base and together both haptics curve away from the base circumference in a spiral direction, each said haptic comprising an outer periphery and an inner periphery with respect to said base; and wherein at least one penetrating member is mounted on said outer periphery of each haptic and is capable of curvilinear penetration upon rotation of said lens system generally about the optical axis of the eye in a first direction and is free to rotate about the eye optical axis in the other direction, and at least two spaced apart penetrating members are mounted on said outer periphery of said other haptic and are capable of curvilinear penetration upon rotation of said lens system generally about the optical axis of the eye in said first direction and is free to rotate about the eye optical axis in said other direction, and wherein said first mentioned manipulating step includes rotating and manipulating said haptics in a direction such that each penetrating member mounted on said haptic penetrates the ciliary sulcus scleral wall and is securely embedded therein so as to securely anchor said haptic system in the posterior chamber of the eye.
